(19) United States
(12) Patent Application Publication
(10) Pub. No.: US 2020/0146890 A1
(43) Pub. Date: May 14, 2020

(54) EYE MASK

(71) Applicant: OCuSOFT, Inc., Rosenberg, TX (US)

(72) Inventors: Troy Smith, Rosenberg, TX (US); Thomas Mason, Rosenberg, TX (US)

(21) Appl. No.: 16/683,660

(22) Filed: Nov. 14, 2019

Related U.S. Application Data
(60) Provisional application No. 62/767,226, filed on Nov. 14, 2018.

(51) Int. Cl. A61F 9/04; A61F 7/02; A45D 44/00

(52) U.S. Cl. CPC A61F 9/04; A61F 7/02; A45D 44/002; A61F 2007/0004; A61F 2007/0231; A61F 2007/023; A61F 2007/0242

(57) ABSTRACT

The invention relates to an eye mask for treating dry eyes and other eye conditions related to blepharitis or meibomian gland dysfunction. The eye mask acts as a warm compress which ensures the eyelids are heated to expedite eyelid expression to balance out the tear film and treat dry eyes. In one embodiment, an eye mask for treating an ocular condition includes: a fabric having a first layer and a second layer opposite the first layer; and a heat diffusing fill material enclosed between the first and the second layers. The first layer and the second layer are stitched together substantially along their edges. The fill material is configured to deliver a moist heat to an ocular surface.

### CROSS-REFERENCE TO RELATED APPLICATIONS

This application claims the benefit of Provisional U.S. Patent Application No. 62/767,226 filed Nov. 14, 2018, entitled “EYE MASK”, the entire content and disclosure of which, both express and implied, is incorporated herein by reference.

### BACKGROUND

The invention relates to an ocular care apparatus and, in particular, to an eye mask for treating dry eyes, blepharitis, meibomian gland dysfunction/disease, allergies and other eye conditions.

The eyes produce tears to stay moist. Tears are necessary for maintaining the health of the front surface of the eye and for providing clear vision. Tears provide lubrication, reduce the risk of eye infection, wash away foreign matter in the eye and keep the surface of the eyes smooth and clear. Excess tears in the eyes flow into small drainage ducts in the inner corners of the eyelids, which drain into the back of the sinuses. However, if the eyes do not make sufficient tears or something affects one or more layers of the tear film, a person can end up with dry eyes. Dry eyes can occur when tear production is decreased, the quality of the tear film is not in balance or drainage is not sufficient. Dry eye is a condition in which a person doesn't have enough quality or quantity of tears to lubricate and nourish the eye. Dry eye is a common and often chronic problem, particularly in older adults.

People with dry eyes either do not produce enough tears or their tears are of a poor quality. People with dry eyes may experience irritated, gritty, scratchy or burning eyes; a feeling of something in their eyes; excess watering; and blurred vision. Advanced dry eyes may damage the front surface of the eye and impair vision.

Treatments for dry eyes aim to restore or maintain the normal amount of tears in the eye to minimize dryness and related discomfort and to maintain eye health. In evaporative dry eyes, the meibomian glands that secrete the lipid layer or outermost layer of the tear film are inflamed and/or capped, so they do not produce the thin lipid layer, but rather have a thick sebum that disrupts the normal quality of the tear film.

Compresses and masks for treating dry eyes are known in the art. Some conventional masks have beads or similar materials which are easily displaced. As such, they do not allow for consistent heat transfer in the target eyelid space when the mask is warmed or heated. These and other conventional masks are configured to be heated in a microwave. Conventional masks may result in a transfer of dirt, oils, and bacteria between uses that then leads to contamination and cross-contamination.

Accordingly, there is a need for a mask which can facilitate and maintain a consistent heat transfer at the target eyelid area as well as protect the user from transfer of dirt, oils, debris, or bacteria that may lead to cross-contamination.

### SUMMARY

The invention relates to an ocular care apparatus for treating dry, scratchy and irritated eyes, blepharitis, meibomian gland dysfunction/disease, allergies and other eye conditions (hereianafter referred to as an “ocular condition”). In one embodiment, the ocular care apparatus includes an eye mask (or, interchangeably, a mask) that can be worn by a person suffering from an ocular condition. The one or more embodiments of the mask are configured to soothe, moisturize and relieve symptoms associated with dry eyes and other ocular conditions.

In one embodiment, an eye mask for treating an ocular condition includes: a fabric having a first layer and a second layer opposite the first layer; and a heat diffusing fill material enclosed between the first and the second layers. The first layer and the second layer are stitched together substantially along their edges. The fill material is configured to deliver a moist heat to an ocular surface.

According to an embodiment, a method for treating dry eyes involves: providing the eye mask having: a first layer and a second layer opposite the first layer and a heat diffusing fill material enclosed between the first and the second layers; heating the mask in a microwave oven for a predetermined time period; and placing the heated mask over a user's eyes. The mask can be left in place for a desired period of time. The eyelids can be cleansed with an eyelid cleanser after the mask has been removed.

In another embodiment, an eye mask for treating an ocular condition includes: (A) a cover, the cover having a fabric comprising: a first layer; a second layer, wherein the first layer and the second layer are stitched together substantially along their edges; and a pocket formed between the first layer and the second layer; and (B) an insert, wherein the insert is configured for placement within the pocket. The insert has a heat diffusing fill material. The fill material is configured to deliver a moist heat to an ocular surface.

According to an embodiment, a method for treating dry eyes involves providing the eye mask having a cover and a removable insert; heating the insert in a microwave oven for a predetermined time period; placing the heated insert within a sleeve in the cover; and placing the cover with the heated inserted over a user's eyes. After use, the mask can be removed and the eyes can be cleaned with an eyelid cleanser.

The fill material can be arranged in a cross-hatch pattern. The fill material can be a desiccant. The fill material is thermally stable and a good thermal conductor. The heated fill material is configured to dissipate a consistent heat to the surface of the eyes. In one embodiment, the fill material includes a plurality of silica beads.

According to another embodiment, a kit for providing heat therapy for the eyes includes an eye mask according to one or more embodiments of the invention and one or more eyelid wipes. The eyelid wipes can be impregnated with an eyelid cleanser. The eyelid cleanser includes a composition having polyhexamethylene biguanide.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the following description.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating a preferred embodiment of the invention and are not to be construed as limiting the invention. In the drawings:

FIGS. 1-3 illustrate an eye mask according to an embodiment of the invention.

FIGS. 4-7 illustrate an eye mask according to another embodiment of the invention.

FIGS. 8-10 illustrate a removable insert according to an embodiment of the invention.

### DETAILED DESCRIPTION

The one or more embodiments of the eye mask are configured as a moist-heat compress for treating symptoms associated with dry eyes and dry, scratchy and irritated eyes. The mask is configured to evenly distribute heat and moisture to the surface of the eyes. The mask includes heat diffusing fill material which are configured to naturally attract moisture from ambient air and release it when the mask is heated in a microwave.

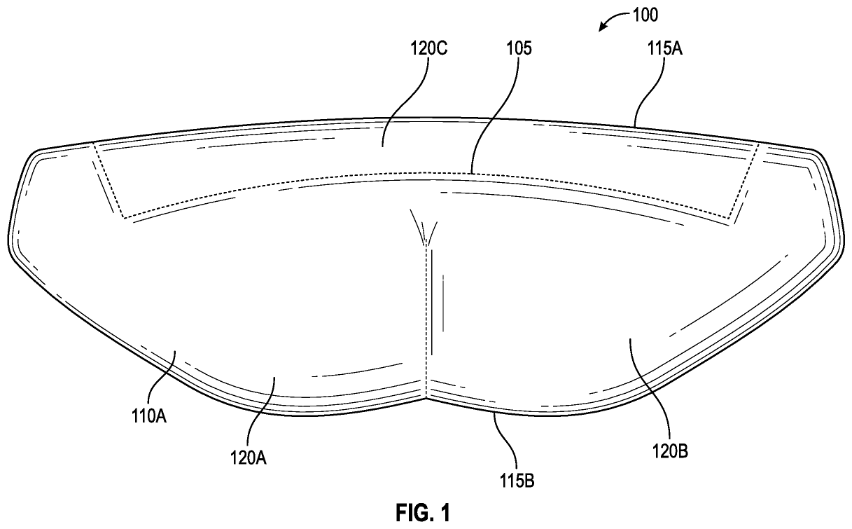

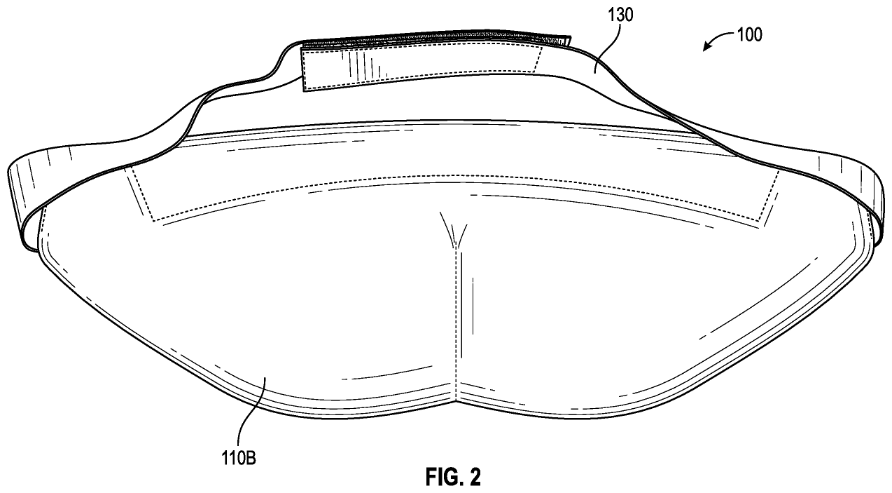

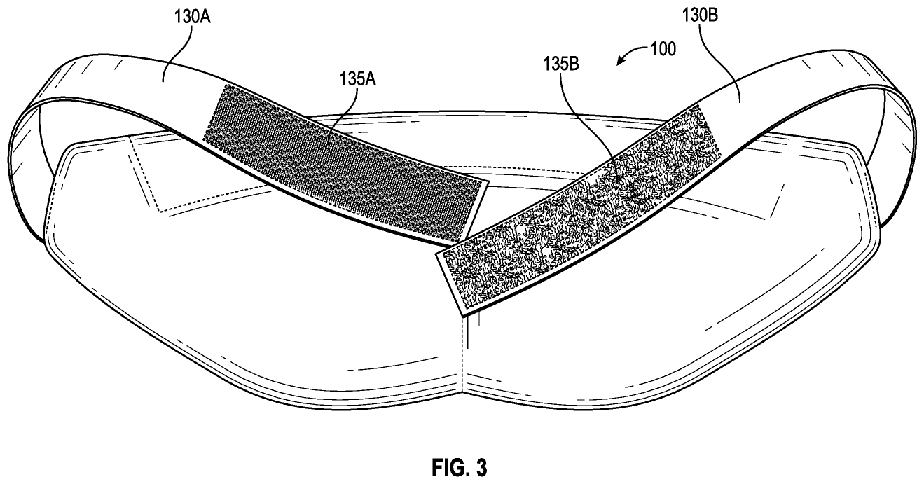

In one embodiment, as shown in FIGS. 1-3, a mask 100 for providing moist heat is provided. As used herein, the term “moist heat” means heat having a moisture content. The mask 100 can be configured for placement over a user's eyes (not shown). The mask 100 can be oval, ovoid, oblong, elliptical or any other desired shape. The mask 100 includes a fabric having a first or a front layer 110A and an opposite second or a back layer 110 B. Each layer 110A, 110B is a mirror image of the other. The fabric is selected to allow moisture from ambient air to be adsorbed by a fill material enclosed between the two layers. The fabric further facilitates heat dissipation from the fill material to the ocular surface. The fabric can be silk, cotton, wool, nylon, velvet, polyester, synthetics or another suitable fabric. The inner surface of each of the layers may include an insulating material.

The two layers 110A, 110B can be stitched together, or otherwise attached, substantially along their edges or peripheries. Each layer 110A, 110B may be connected to the other layer with one or more rows of stitch points/stitching. The stitching can be in the form a pre-determined pattern. For example, the pattern may include a plurality of intersecting parallel lines. The thread used for stitching can be made of any material known in the art. Preferably, the thread may be made of silk, cotton, polyester, synthetics or another suitable material. A fill material is distributed and completely enclosed within the stitched front and back layers 110A, 110B.

A top edge 115A of the mask can be arcuate. A bottom edge 115B is arcuate with an indention where it would fit over the bridge of the nose. One or more rows of stitch points define at least three substantially distinct sections 120A, 120B and 120C in the front layer 110A and the back layer 110B. The fill material is enclosed within each of the sections.

A first section 120C is located substantially proximal the top edge 115A. The first section 120C is elongate and extends along a horizontal axis. The first section 120C can be configured to be placed above a user's eyes, such as, over the user's eyebrows and the forehead (not shown). The first section 120C is substantially segregated from the second and third sections 120A, 120B by one or more rows of stitch points 105. The fill material enclosed within the first section 120C is substantially isolated from the fill material contained in the second and third sections 120A, 120B. A second section 120A is configured to be placed over a first eye while a third section 120B is configured to be placed over a second eye. The sections are configured to ensure optimal thermal diffusivity.

The mask 100 can include a flexible strap component 130. The strap component 130 can be elasticated for adjustability and ease of use. The strap component 130 can include a first strap member 130A and a second strap member 130B. The first strap member 130A is interposed and stitched between the first layer 110A and the second layer 110B along a first side while the second strap member 130B is interposed and stitched between the first layer 110A and the second layer 110B along a second side that is opposite the first side. Each strap member 130A, 130B can include one or more fastening elements. In one aspect, a first surface of each strap member 130A, 130B can include pads having mating hook-and-loop type engaging fasteners 135A, 135B. However, the strap members can also include buckles, hooks and other fastening elements to connect the first strap member 130A to the second strap member 130B.

The strap component 130 is configured to be fitted around/fastened to the back of the head. By adjusting the strap component 130, a comfortable pressure can be applied between an exterior surface of the second layer 110B and the eyes of the user.

In one embodiment, the inside of the mask 100 can contain one or more interchangeable or adjustable bands (not shown). The bands are sewn to the backside/inside of the mask in a horizontal or vertical pattern. The bands are configured to hold a lint-free cloth/fabric. The lint-free cloth may include a rayon pad, a rayon and polypropylene fabric blend, cotton, or other like lint-free materials. The lint-free cloth can comprise a textured or smoothed surface. The lint-free cloth can be replaced or interchanged to prevent cross-contamination.

The lint-free cloth can be sprayed with one or more anti-bacterial/anti-microbial solutions prior to placing on the closed eyelids and interchanged between uses. For example, the lint-free cloth may be sprayed or moistened with a solution containing consisting essentially of ≥99% water and ≤0.02% hypochlorous acid.

A fill material (not shown) is contained within the enclosure formed by the stitched front and back layers 110A, 110B. The fill material can include a granular material, fabric, a film or a foam or any other material that has one or more desired characteristics. The desired characteristics can include, for example, moisture adsorption, good thermal conductivity and compressibility. Furthermore, the fill material may include a one or more types of fill materials.

In one embodiment, the fill material can be a desiccant configured to absorb moisture from air by physical adsorption. The fill material can be selected from a group consisting of silica, activated alumina, clay, synthetic materials, polymers and activated charcoal and combinations thereof. Importantly, the fill material is thermally stable, and it is a good thermal conductor. The fill material is configured to retain a consistent heat for at least a short period of time. The fill material can be configured to dissipate or deliver a consistent moist heat to an ocular surface.

In another embodiment, the fill material may include dried whole peas, buckwheat groats, oats, walnut shells, flaxseeds, dried pinto bean, or similar types of materials or derivatives of these materials.

In an exemplary embodiment, the fill material is a silica gel-based material. Silica gel is a chemically inert, non-toxic material composed of amorphous silicon dioxide. It has an internal network of interconnecting microscopic pores. Silica gel can adsorb or desorb moisture until a vapor pressure equilibrium is achieved with the relative humidity of ambient air.

The silica gel-based material may have any of the physical forms normally available. For example, the form can be irregular granules or approximately spherical silica beads, which are called spherical or beaded silica gel. The selection of the size, shape and quantity of the silica beads or other fill material is based on known scientific principles.

In an embodiment, the fill material may be distributed in a designated pattern. The pattern can be a cross-hatch pattern, a basket weave pattern or any other suitable pattern. This can facilitate an even distribution of the fill material and also optimize the dissipation of moist heat over the eyes.

According to an embodiment, a method of treating an ocular condition involves providing the mask 100. The mask 100 can be heated for a predetermined time period in a heating source, such as, a microwave oven. The predetermined time period can include from about 15 to about 30 seconds. Heating the mask 100 causes the enclosed fill material to be heated. In one embodiment, the mask can be initially heated for 20 seconds and, if necessary, it can be heated by an additional 5-10 seconds.

Care is taken to avoid heating the mask for more than 30 seconds in order to avoid overheating or damaging the fill material. The temperature of the mask 100 is checked, for example, by touching it, before it is worn by the user to ensure that the temperature is within acceptable limits for placement over the eyes. If the mask 100 feels too hot, the user should wait until some of the heat is dissipated and the user can tolerate the heat.

If the temperature is acceptable to the user, it can be worn by placing the back layer 110B of the mask over the eyes/ocular surface. The mask 100 can then be securely and comfortably fastened to the back of the head by means of the adjustable and flexible strap component 135. The heated fill material is configured to slowly diffuse heat and the adsorbed moisture as a moist heat through the fabric layers. The heat is diffused over the surface of the eyes such that it can relieve, moisturize and relieve dry eyes and other ocular conditions. The mask 100 can be placed over the eyes for a desired time period. For optimal heat diffusion, the mask 100 can be placed over the closed eyes for about 5-20 minutes, or as prescribed by a medical professional. The mask 100 is also configured to be cooled, if needed, to treat inflammation of the ocular surface.

The heated fill material retains the heat and ensures that heat is transferred consistently to the targeted eyelids. The heat can melt the sebum of the meibomian glands. This allows the user to easily massage or express these glands manually to secrete thin lipids to help balance the tear film and treat the symptoms of dry eyes.

After use for a desired amount of time, the mask 100 is removed. Silica beads have an extended life in terms of the ability to adsorb or desorb moisture. Therefore, the mask 100, containing the silica beads as fill material, can be reconditioned and reused. If the mask 100 is reused within 30 minutes, the heating time can be reduced by about half the initial heating time.

According to an embodiment, a method of decontaminating the mask 100 is disclosed. If the mask 100 is used multiple times it may get contaminated. As such, it may be necessary to decontaminate the mask in order to prevent infection of the eyes/eyelids. The mask 100 and, in particular, the back layer 110B that contacts the eyes can be sprayed or wiped with a disinfecting solution, such as, OCuSOFT® Hypochlor, consisting essentially of ≥99% water and ≤0.02% hypochlorous acid. The disinfecting solution may be in the form of a gel or a spray. In one or more embodiments, a dry eye mask kit can include the mask 100 and a hypochlorous acid spray and/or one or more wipes impregnated with hypochlorous acid.

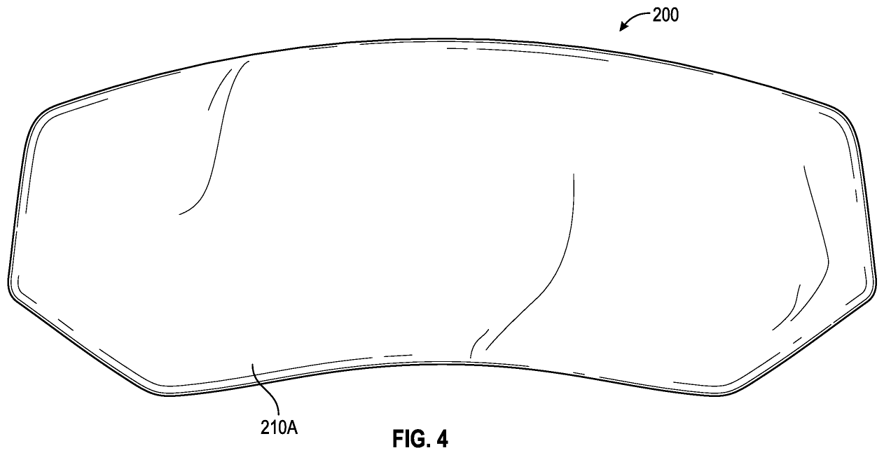

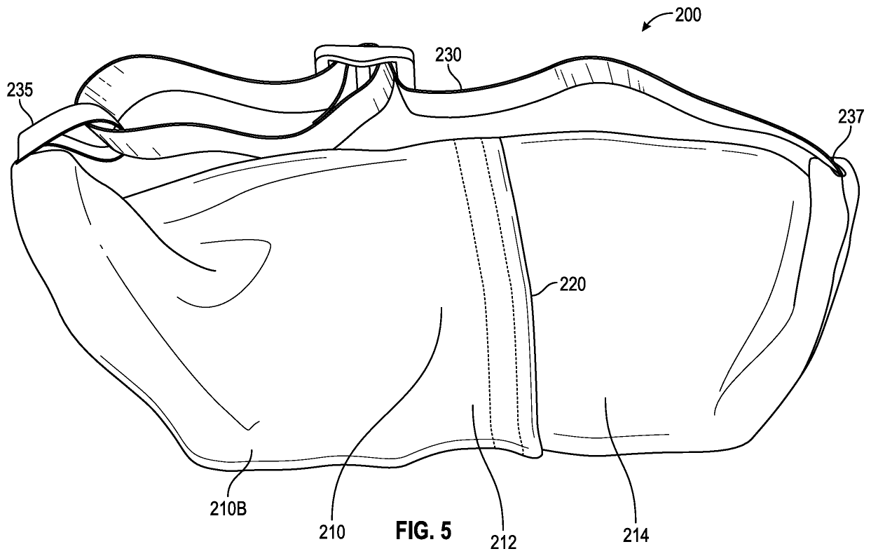

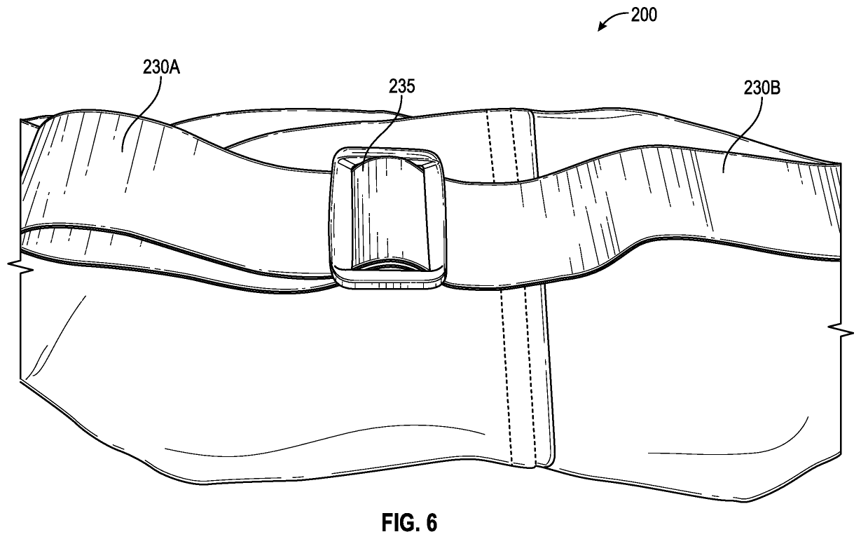

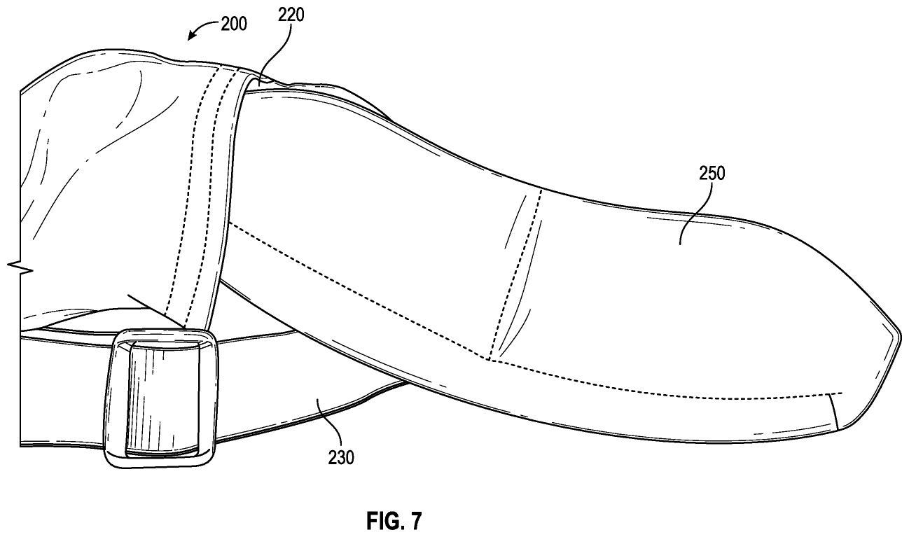

FIGS. 4-7 illustrate another embodiment of an eye mask 200 for providing moist heat. The mask 200 can be worn by a user with an ocular condition. The mask 200 can be configured for placement over a user's eyes (not shown). The mask 200 can be oval, ovoid, oblong, elliptical or any other desired shape. The mask 200 includes a cover 210 and a removable insert 250. The insert 250 includes one or a combination of heat diffusing fill materials.

The cover 210 includes a fabric having a first or a front layer 210A and an opposite second or a back layer 210B. Each layer 210A, 210B is a mirror image of the other. The fabric is selected to allow moisture from ambient air to be adsorbed by a fill material enclosed in an insert. The fabric further facilitates heat dissipation from the fill material to the ocular surface. The fabric can be silk, cotton, wool, nylon, velvet, polyester, synthetics or another suitable fabric. The top and bottom edges of the mask 200 can have a generally arcuate shape. The bottom edge of the cover 210 can have an indention where it would fit over the bridge of the nose. The inner surface of each of the layers may include an insulating material.

The two layers 210A, 210B can be stitched together, or otherwise attached, at their edges or peripheries. Each layer 210A, 210B may be connected to the other layer with multiple rows of stitch points. The stitching can be in the form a pre-determined pattern. For example, the pattern may include a plurality of intersecting parallel lines. The thread used for stitching can be made of any material known in the art. Preferably, the thread may be made of silk, cotton, polyester, synthetics or another suitable material.

The back layer 210B includes two substantially superposed fabric halves 212 and 214. In one embodiment, the two halves 212, 214 define an elongate access opening 220 therebetween. The access opening 220 is configured as a pocket for receiving one or more removable inserts 250 between the first layer 210A and the second layer 210B. In certain embodiments, there may be a single sleeve for a single insert or two sleeves with one sleeve accessible from each side of the mask.

The cover 210 can include a flexible strap component 230. The strap component 230 can be elasticated for adjustability and ease of use. The strap component 230 can include a first portion 230A and a second portion 230B. The first portion 230A is inserted within or looped around a strap insertion member 235. The ends of the strap insertion member 235 can be interposed and stitched between the first layer 210A and the second layer 210B along a first side. The strap insertion member 235 can be made of any suitable fabric. The second portion 230B of the strap component 230 is interposed and stitched between the first layer 210A and the second layer 210B along a second side 237. The strap component 230 can include fastening elements. In one aspect, the strap component 230 can include a buckle-type fastener 235. However, the strap component can also include hooks, mating hook-and-loop type engaging fasteners and other fastening elements.

The strap component 230 is configured to be fitted around/fastened to the back of the user's head. The strap component 230 can provide a means for adjusting the tension exerted on the eyes by the back layer 210B of the mask.

An insert, for the purposes of this disclosure can include, without limitations, a cushion, pad, pod or pillow. The insert can substantially completely enclose one or a combination of fill materials. The fill material can be fabric, beaded silica gel or any other material that has one or more desired characteristics. The desired characteristics can include, for example, moisture adsorption, good thermal conductivity and compressibility. In another embodiment, the insert can include a relatively thick piece of a fabric or material having good thermal conductivity.

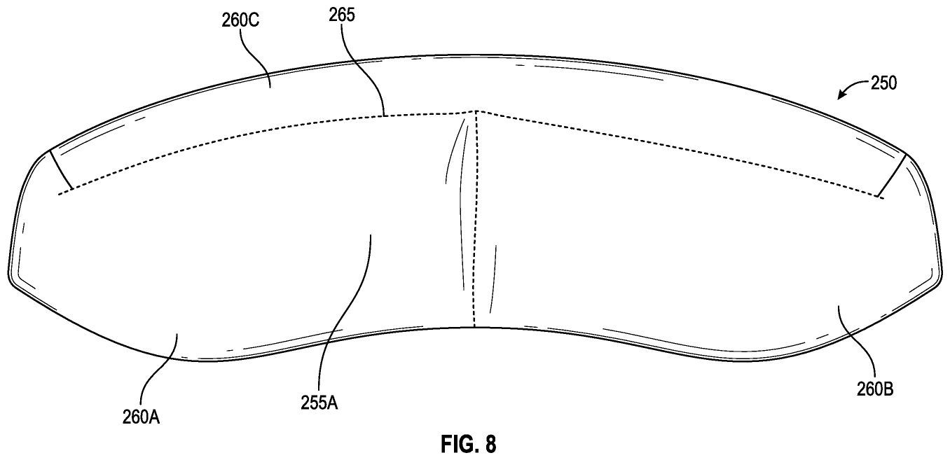

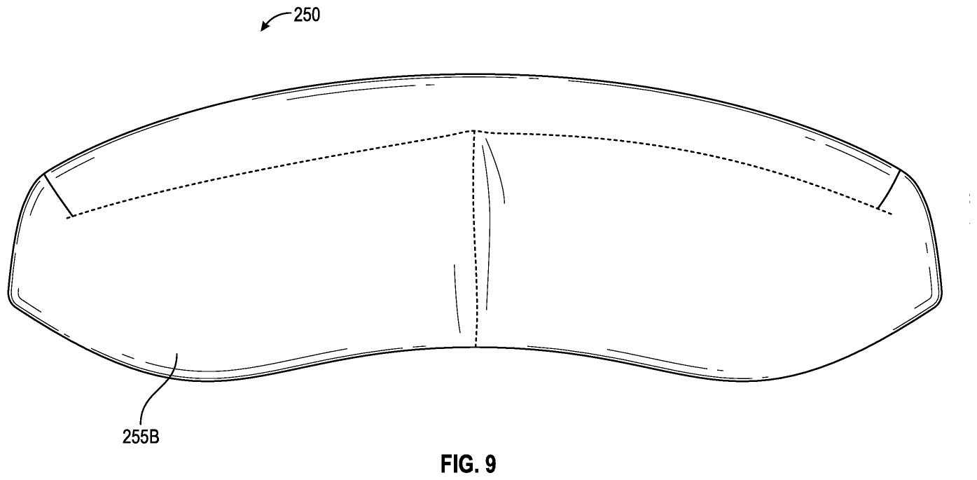

FIGS. 8-10 illustrate an exemplary insert 250. The insert 250 can include a fill material (not shown) which is configured adsorb moisture from ambient air. The fill material is a good thermal conductor. The insert 250 can be heated and placed within the pocket 220 prior to use. The heated insert is configured to diffuse moist heat uniformly over an ocular surface thereby providing the user/wearer relief from the ocular condition. After use, the insert 250 can be removed from the cover 210 and the cover can be conveniently hand- or machine-washed to ensure optimal ocular hygiene.

The insert 250 includes a fabric having a front surface 255A and a back surface 255B. The fabric is configured to allow moisture from ambient air to be adsorbed by the fill material. The fabric can be silk, cotton, wool, nylon, velvet, polyester, synthetics or another suitable fabric. The two surfaces 255A, 255B can be stitched together, or otherwise attached, at their edges or peripheries. Each surface 255A, 255B may be connected to the other surface with multiple rows of stitch points. The stitching can be in the form a pre-determined pattern. For example, the pattern may include a plurality of intersecting parallel lines. The thread used for stitching can be made of any material known in the art. Preferably, the thread may be made of silk, cotton, polyester, synthetics or another suitable material.

A top edge of the insert can be arcuate. A bottom edge is arcuate with an indention corresponding to indentation on the bottom of the cover 210. One or more rows of stitch points define at least three substantially distinct sections 260A, 260B and 260C in the front surface 255A and the back surface 255B. The fill material is enclosed within each of the sections.

A first section 260C is located substantially proximal a top edge of the insert 250. The first section 260C is elongate and extends along a horizontal axis. The first section 260C is substantially segregated from the second and third sections 260A, 260B by one or more rows of stitch points 265. The fill material enclosed within the first section 260C is substantially isolated from the fill material contained in the second and third sections 260A, 260B. The sections are configured to ensure optimal thermal diffusivity.

The insert 250 can be placed within the pocket 220 such that the back surface 255B of the insert is adjacent the back layer 210B and the front surface 255A of the insert is adjacent the front layer 210A of the cover 210. The insert 250 is sized such that, when it is inserted within the sleeve 220, it forms a snug fit with the inside sidewalls of the cover 210.

The insert 250 can be oval, ovoid, oblong, elliptical or any other desired shape. Preferably, the shape of the insert 250 matches the contours of the cover 210.

A fill material (not shown) is contained within an enclosure formed by the stitched front and back layers 255A, 255B. The fill material can include a granular material, fabric, a film or a foam or any other material that has one or more desired characteristics. The desired characteristics can include, for example, moisture adsorption, good thermal conductivity and compressibility. Furthermore, the fill material may include a one or more types of fill materials.

In one embodiment, the fill material can be a desiccant configured to absorb moisture from air by physical adsorption. The fill material can be selected from a group consisting of silica, activated alumina, clay, synthetic materials, polymers and activated charcoal and combinations thereof. Importantly, the fill material is thermally stable, and it is a good thermal conductor. The fill material is configured to retain a consistent heat for at least a short period of time. The fill material can be configured to dissipate or deliver a consistent moist heat to an ocular surface.

In another embodiment, the fill material may include dried whole peas, buckwheat groats, oats, walnut shells, flaxseeds, dried pinto bean, or similar types of materials or derivatives of these materials.

In an exemplary embodiment, the fill material is a silica gel-based material. Silica gel is a chemically inert, non-toxic material composed of amorphous silicon dioxide. It has an internal network of interconnecting microscopic pores. Silica gel can adsorb or desorb moisture until a vapor pressure equilibrium is achieved with the relative humidity of ambient air.

The silica gel-based material may have any of the physical forms normally available. For example, the form can be irregular granules or approximately spherical silica beads, which are called spherical or beaded silica gel. The selection of the size, shape and quantity of the silica beads or other fill material is based on known scientific principles.

In another embodiment, the insert 250 can include a packet (not shown) filled with saturated liquid sodium acetate, sodium thoisulfate, or a similar solution with a releasable supersaturated or crystalline solution. When the packet is squeezed to expose the supersaturated solution or crystalline salt, the spontaneous crystallization of the salt begins. The crystallization of salts such as sodium acetate or sodium thiosulfate are exothermic and release heat. The crystallization process can be sped up by mixing or massaging the packet to further mix the crystals with the saturated solution.

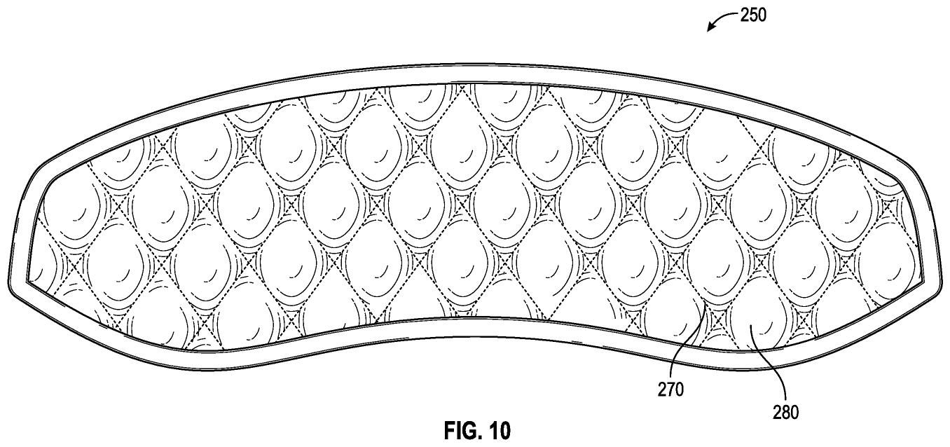

As shown in FIG. 10, an inside or outside surface of the insert 250 can include stitch points arranged in a predetermined pattern, such as, a cross hatch pattern 270. The fill material is retained within the interstitial spaces 280. This facilitates optimal fill material distribution for targeting the ocular surface along with providing consistent moist-heat transfer to the eyes.

In another embodiment, an inside and/or outside surface of the insert 250 can include one or more pouches or pockets. The pockets can be filled with the fill material. The filled pockets can be completely or partially sealed by stitch points. The pockets can be arranged in one or more patterns. For example, the pockets can be arranged in a cross-hatch pattern. However, the pockets can be arranged in any other suitable pattern, including a basket weave pattern. The fill material is securely held in place or retained within each of the cross-hatch pockets to facilitate optimal moist-heat transfer to the eyes.

In an embodiment, a method of treating dry eyes involves providing the mask 200. The insert 250 is removed from the cover 210. The insert 250 is heated for a predetermined time period. For example, the insert is heated from about 15 to about 30 seconds in a heating source, such as, a microwave oven. Heating the insert 250 causes the enclosed fill material to be heated. In one embodiment, the insert 250 can be initially heated for 20 seconds and, if necessary, the it can be further heated for another 5-10 seconds. The heated insert 250 is configured to retain heat and to diffuse the heat consistently to the eyes along with the adsorbed moisture. The heated insert 250 can provide equal distribution of moist heat over the eye area for relief from dry eye symptoms. The insert 250 is also configured to be cooled, if needed, to treat inflammation of the ocular surface.

Care is taken to avoid heating the insert 250 for more than 30 seconds to avoid overheating the fill material. The temperature of the heated insert is checked, for example, by touch testing, for tolerance prior to inserting it within the pocket 220 of the cover 210. If the insert 250 feels too hot, the user should wait until the temperature is acceptable before placing it within the cover 210. If the temperature is acceptable to the user, insert 250 can be placed within the pocket.

The back layer 210B of the cover with the heated insert 250 can be placed over the closed eyes/ocular surface. The mask 200 can then be securely fastened to the back of the head by means of the adjustable and flexible strap component 235 so that the mask 200 lies comfortably over the ocular surface. The heated fill material is configured to slowly diffuse heat and release the adsorbed moisture. The moist heat is diffused over the surface of the eyes such that it can relieve, moisturize and relieve the symptoms associated with dry eyes and other ocular conditions. The mask 200 is left in place for a desired period of time. For optimal heat diffusion, the mask 200 can be placed over the closed eyes for about 5-20 minutes, or as prescribed by a medical professional.

The heat from the mask 200 can melt the sebum of the meibomian glands. The allows the user to easily massage or express these glands manually to secrete thin lipids to help balance the tear film and treat dry eyes.

After use for a desired amount of time, the mask 200 is removed. Silica beads have an extended life in terms of the ability to adsorb or desorb moisture. Therefore, the insert 250, containing the silica beads as the insert 250 fill material, can be reconditioned and reused. If the mask 200 is reused within 30 minutes, the insert 250 heating time can be reduced by about half the initial heating time.

According to an embodiment, the insert 250 can be removed from the cover 210 and the cover can be conveniently washed before reusing the mask 200. The cover 210 can be configured to be washed by hand or machine after the insert 250 is removed. This can ensure that cover is free from contaminants. This can promote better eye hygiene. The insert 250 can also be hand-washed, if necessary, and allowed to air dry before it is re-used.

According to another embodiment, a method of decontaminating the insert 250 is disclosed. The insert 250 can be sprayed or wiped with a disinfecting solution, such as, OCuSOFT® Hypochlor consisting essentially of ≥99% water and ≤0.02% hypochlorous acid. The disinfecting solution may be in the form of a gel or a spray.

After the mask 100/200 has been removed from the ocular surface or after application of the mask 100/200 to the ocular surface, the eyelids can be cleansed with an eyelid cleanser to remove oil, debris or other contaminants. The eyelid cleanser can be selected from a group consisting of a solution, a foam, a gel, a spray, a lotion, a suspension, an emulsion, an ointment and combinations thereof. In one embodiment, the eyelids are cleansed with a wipe impregnated with an eyelid cleanser containing polyhexamethylene biguanide.

In one embodiment, the eyelid cleanser includes a composition having: polyhexamethylene biguanide; 1,2-hexanediol; and 1,2-octanediol and one or more surfactants. The surfactants may be present in an amount effective to control a pH of the first composition within a desired pH range. U.S. Pat. No. 7,951,387, herein incorporated by reference in its entirety, describes such a composition having polyhexamethylene biguanide; 1,2-hexanediol; and 1,2-octanediol.

In yet another embodiment, the eyelid cleanser includes a composition having: purified water; PEG-80 sorbitan laurate; sodium trideceth sulfate; PEG-150 distearate; sodium lauroamphoacetate; cocamidopropyl hydroxysultaine; sodium laureth-13 carboxylate; sodium chloride; PEG-15 cocopolyamine; polyhexamethylene biguanide; potassium sorbate; 1,2 hexanediol; and caprylyl glycol. U.S. Pat. No. 9,278,079, herein incorporated by reference in its entirety, describes such a composition.

In another embodiment, the eyelid cleanser includes a composition comprising: (a) a mixture of antimicrobial agents that substantially eliminates at least seven bacterial strains, wherein the mixture includes polyaminopropyl biguanide, a 1,2-glycol, and a sphingoid lipid; (b) a blended surfactant solution that maintains the first ophthalmic composition at a pH between 5.5 and 7.5, wherein the blended surfactant solution has a foaming ability; (c) a foam stabilizer; and (d) a moisturizer. U.S. Pat. No. 9,833,399, herein incorporated by reference in its entirety, describes such a composition.

In one embodiment, a dry eye mask kit is provided. The kit includes the mask 100/200. The kit can also include one or more wipes impregnated/pre-moistened with a therapeutically effective amount of the eyelid cleanser disclosed herein. The kit can include between 1-100 wipes. The wipes may be sealed or wrapped within suitable packaging. The kit or the mask(s) can also include instructions for using the mask(s) 100/200.

Advantageously, the masks 100, 200 do not contain any added chemicals and are filled with silica or other beads that do not irritate the eyes. The masks 100, 200 are configured to be soft, gentle to the eye and easy to use.

As used herein, the term “therapeutically effective amount” of a composition refers to an amount sufficient to cleanse the eyelid.

As used herein, “comprising” means the elements recited, or their equivalent, plus any other element or elements which are not recited. The terms “having,” “including,” and “comprised of” are also to be construed as open ended unless the context suggests otherwise. As used herein, “consisting essentially of” means that the invention may include elements in addition to those recited in the claim, but only if the additional elements do not materially alter the basic and novel characteristics of the claimed invention.

It will be understood that, although the terms first, second, front, back etc may be used herein to describe various elements, materials, layers, and/or surfaces, these elements, materials, layers, and/or surfaces should not be limited by these terms. These terms are only used to distinguish one element, material, layer or surface from another element, material, layer or surface. Thus, a first element, material, layer or surface discussed above could be termed a second element, material, layer or surface without departing from the teachings of the present invention.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

1. An eye mask for treating an ocular condition, comprising:
a fabric having a first layer and a second layer opposite the first layer; and
a heat diffusing fill material enclosed between the first and the second layers,
wherein the first layer and the second layer are stitched together substantially along their edges, and
wherein the fill material is configured to deliver a moist heat to an ocular surface.

2. The eye mask according to claim 1, wherein one or more rows of stitch points define at least three substantially distinct sections in the front layer and the back layer.

3. The eye mask according to claim 2, wherein a first section is located substantially along a horizontal axis.

4. The eye mask according to claim 3, wherein the first section is substantially segregated from a second section and a third section by one or more rows of stitch points.

5. The eye mask according to claim 4, wherein the fill material enclosed in the first section is substantially isolated from the fill material enclosed in the second section and the third section.

6. The eye mask according to claim 1, wherein the fill material comprises a thermally conductive material.

7. The eye mask according to claim 1, wherein the fill material comprises a plurality of silica beads.

8. The eye mask according to claim 1, wherein the eye mask is configured to treat dry eyes.

9. The eye mask according to claim 1, further comprising an adjustable strap component, wherein the strap component comprises:
a first strap member interposed between the first layer and the second layer along a first side of the mask; and
a second strap member interposed between the first layer and the second layer along a second side of the mask,
wherein each strap member includes one or more fastening elements.

10. An eye mask for treating an ocular condition, comprising:
(A) a cover, the cover having a fabric comprising:
a first layer;
a second layer, wherein the first layer and the second layer are stitched together substantially along their edges; and
a pocket formed between the first layer and the second layer; and
(B) an insert, wherein the insert is configured for placement within the pocket, wherein the insert comprises a heat diffusing fill material, and wherein the fill material is configured to deliver a moist heat to an ocular surface.

11. The eye mask according to claim 10, wherein the fill material is arranged in a cross-hatch pattern.

12. The eye mask according to claim 10, wherein the fill material comprises a thermally conductive material.

13. The eye mask according to claim 10, wherein the insert is removable, and further wherein the insert is configured to be heated in a microwave.

14. The eye mask according to claim 10, wherein the cover is configured to be hand- or machine-washed.

15. A kit for providing heat therapy for the eyes comprising:
(a) eye mask according to claim 1; and
(b) one or more eyelid wipes, wherein the eyelid wipe is impregnated with an eyelid cleanser.

16. The kit according to claim 15, wherein the eyelid cleanser includes a composition having polyhexamethylene biguanide.

17. A kit for providing heat therapy for the eyes comprising:
(a) an mask according to claim 10; and
(b) one or more eyelid wipes, wherein the eyelid wipe is impregnated with an eyelid cleanser.

18. The kit according to claim 17, wherein the eyelid cleanser includes a composition having polyhexamethylene biguanide.

19. A method for treating dry eyes, comprising:
(a) providing an eye mask according to claim 1;
(b) heating the mask in a microwave oven for a predetermined time period; and
(c) placing the heated mask over a user's eyes.

20. A method for treating dry eyes, comprising:
(a) providing an eye mask according to claim 10;
(b) heating the insert in a microwave oven for a predetermined time period;
(c) placing the heated insert within the pocket; and
(d) placing the cover with the heated inserted over a user's eyes.
